(19) United States
(12) Patent Application Publication
(10) Pub. No.: US 2023/0400357 A1
(43) Pub. Date: Dec. 14, 2023

(54) ELECTRONIC DEVICE AND METHOD OF ESTIMATING BODY TEMPERATURE USING THE SAME

(71) Applicant: SAMSUNG ELECTRONICS CO., LTD., Suwon-si (KR)

(72) Inventors: So Young LEE, Suwon-si (KR); Sang Kyu KIM, Yongin-si (KR); Bok Soon KWON, Seoul (KR); Sung Ho KIM, Suwon-si (KR); Ho Taik LEE, Yongin-si (KR); Hong Soon RHEE, Suwon-si (KR)

(73) Assignee: SAMSUNG ELECTRONICS CO., LTD., Suwon-si (KR)

(21) Appl. No.: 17/966,397

(22) Filed: Oct. 14, 2022

(30) Foreign Application Priority Data
Jun. 13, 2022 (KR) 10-2022-0071740
Sep. 29, 2022 (KR) 10-2022-0124169

(51) Int. Cl. G01K 1/02; G01K 1/024; G01K 13/20

(52) U.S. Cl. CPC G01K 1/026; G01K 1/024; G01K 13/20

(57) ABSTRACT

An electronic device may include: a first temperature sensor configured to measure a first temperature of a surface at a body measurement location of a user; a second temperature sensor spaced apart from the first temperature sensor and configured to measure a second temperature inside a main body of the electronic device; a third temperature sensor disposed further away from the first temperature than the second temperature sensor and configured to measure a third temperature inside the main body; and a processor configured to estimate a core temperature at the body measurement location based on the first temperature, the second temperature, and the third temperature; estimate an ambient temperature outside the main body based on the second temperature and the third temperature; and estimate a body temperature of the user based on the core temperature at the body measurement location and the ambient temperature outside the main body.

### CROSS-REFERENCE TO RELATED APPLICATION(S)

This application claims priority from Korean Patent Application No. 10-2022-0071740, filed on Jun. 13, 2022 and Korean Patent Application No. 10-2022-0124169, filed on Sep. 29, 2022 in the Korean Intellectual Property Office, the disclosure of which is incorporated herein by reference in its entirety.

### BACKGROUND

### 1. Field

Apparatuses and methods consistent with example embodiments relate to estimating an ambient temperature and a body temperature using an electronic device.

### 2. Description of the Related Art

Generally, body temperature is one of four vital signs and has very important clinical significance. A body temperature sensor may be applied to various applications, such as checking infections in patients, thermal side effects of medications, or time of ovulation in women, and the like. However, there is a difference between temperature of peripheral parts of the body, e.g., wrist temperature or skin temperature, and core body temperature to be measured, and particularly the temperature of the peripheral parts of the body may vary greatly depending on outside temperature such as air temperature. Accordingly, it may be difficult to measure the core body temperature at a high accuracy by using a wearable device for measuring the temperature of the peripheral parts. A general body temperature sensor may be classified into a contact type sensor and a non-contact type sensor. Examples of the contact type sensor may include a sensor for detecting a change in electrical resistance, such as a Resistance Temperature Detector (RTD), a thermistor, etc., a thermocouple for detecting electromotive force, and the like. Further, examples of the non-contact type sensor may include a thermopile, a micro-bolometer, etc., which measure body temperature by detecting infrared rays radiating from a body surface. A general body temperature measuring technology is significantly affected by a change in environment factors affecting heat transfer, such as a change in external ambient temperature, humidity, air flow, and the like.

### SUMMARY

According to an aspect of the present application, an electronic device may include: a first temperature sensor configured to measure a first temperature of a surface at a body measurement location of a user; a second temperature sensor spaced apart from the first temperature sensor and configured to measure a second temperature inside a main body of the electronic device; a third temperature sensor disposed further away from the first temperature than the second temperature sensor and configured to measure a third temperature inside the main body; and a processor configured to: estimate a core temperature at the body measurement location based on the first temperature, the second temperature, and the third temperature; estimate an ambient temperature outside the main body based on the second temperature and the third temperature; and estimate a body temperature of the user based on the core temperature at the body measurement location and the ambient temperature outside the main body.

The processor may be further configured to: obtain a heat loss from a body reference location to the body measurement location, and estimate the body temperature by correcting the core temperature at the body measurement location based on the heat loss.

The processor may be further configured to obtain the heat loss based on a difference between the first temperature and the ambient temperature outside the main body.

The electronic device may further include a pulse wave sensor including a light source and a detector, and configured to measure a pulse wave signal of the user, wherein the processor may be further configured to: estimate a first heat flux based on a difference between the first temperature and the second temperature; estimate a skin blood flow volume based on the pulse wave signal; and estimate the core temperature at the body measurement location based on the first heat flux, the first temperature, and the skin blood flow volume.

The processor may be further configured to estimate the core temperature at the body measurement location by combining a ratio between the estimated first heat flux and the skin blood flow volume with the first temperature.

The processor may be further configured to: estimate a second heat flux based on a difference between the second temperature and the third temperature; and estimate the ambient temperature outside the main body by combining the second heat flux with the third temperature.

The processor may be further configured to correct the second heat flux based on a resistance value of a thermally conductive material disposed between the second temperature sensor and the third temperature sensor, a resistance value of a thermally conductive material disposed between the third temperature sensor and the surface of the main body, and a resistance value of the surface of the main body.

At least one of the first temperature sensor, the second temperature sensor, and the third temperature sensor may be a thermistor.

The first temperature sensor may be disposed at a vertical distance of 5 mm or less from a contact surface between the main body and the user.

The third temperature sensor may be disposed at a vertical distance of 10 mm or less below the surface of the main body.

A distance between the first temperature sensor and the second temperature sensor may be 10 mm or less, and a distance between the third temperature sensor and the second temperature sensor is 50 mm or less.

The electronic device may further include a display configured to output at least one of the first temperature, the second temperature, the third temperature, the core temperature at the body measurement location, and the ambient temperature outside the main body.

According to another aspect of the present disclosure, a method of estimating body temperature using an electronic device, may include: by using a first temperature sensor, measuring a first temperature of a surface at a body measurement location; by using a second temperature sensor spaced apart from the first temperature sensor, measuring a second temperature inside a main body of the electronic device; by using a third temperature sensor disposed further away from the first temperature than the second temperature sensor, measuring a third temperature inside the main body; estimating a core temperature at the body measurement location based on the first temperature and the second temperature; estimating an ambient temperature outside the main body based on the second temperature and the third temperature; and estimating a body temperature of a user based on the core temperature at the body measurement location and the ambient temperature outside the main body.

The estimating of the body temperature of the user may include: obtaining a heat loss from a body reference location to the body measurement location; and estimating the body temperature by correcting the core temperature at the body measurement location based on the heat loss.

The estimating of the body temperature of the user may include: obtaining the heat loss based on a difference between the first temperature and the ambient temperature outside the main body.

The estimating of the core temperature at the body measurement location may include: estimating a first heat flux based on the first temperature and the second temperature; and estimating the core temperature at the body measurement location based on the first heat flux, the first temperature, and a skin blood flow volume measured by a pulse wave sensor.

The estimating of the ambient temperature outside the main body may include: estimating a second heat flux based on a difference between the second temperature and the third temperature; and estimating the ambient temperature outside the main body by combining the second heat flux with the third temperature.

The estimating of the ambient temperature outside the main body may include: correcting the second heat flux based on a resistance value of a thermally conductive material disposed between the second temperature sensor and the third temperature sensor, a resistance value of a thermally conductive material disposed between the third temperature sensor and the surface of the main body, and a resistance value of the surface of the main body.

According to another aspect of the present disclosure, a wearable device may include: a main body; and a strap connected to the main body, wherein the main body may include a first temperature sensor configured to measure a skin temperature of a user, a second temperature sensor configured to measure a first internal temperature of the main body, a third temperature sensor configured to a second internal temperature of the main body, wherein the second temperature sensor is disposed between the first temperature sensor and the third temperature sensor in a thickness direction of the main body; and a processor configured to estimate a core temperature at a body measurement location based on the skin temperature and the first internal temperature, estimate an ambient temperature outside the main body based on the first internal temperature and the second internal temperature of the main body, and estimate a body temperature of the user based on the core temperature at the body measurement location and the ambient temperature outside the main body.

The wearable device may further include: a display; a sensor circuit board to which the first temperature sensor and a PPG sensor are connected; and a main circuit board to which the processor is connected and disposed between the sensor circuit board and the display, wherein the second temperature sensor may be disposed between the sensor circuit board and the main circuit board, and the third temperature sensor is disposed between the main circuit board and the display.

According to another aspect of the present disclosure, there is provided a wearable device including: a main body; a strap connected to both ends of the main body; first, second, and third temperature sensors disposed at different distances from a body contact surface, the first temperature sensor configured to measure a first temperature, the second temperature sensor configured to measure a second temperature, and the third temperature sensor configured to measure a third temperature; and a processor which, when the main body is worn, is configured to estimate a first heat flux based on the first temperature and the second temperature, to estimate a core temperature at a body measurement location based on the estimated first heat flux and the first temperature, to estimate a second heat flux based on the second temperature and the third temperature, to estimate an ambient temperature outside the main body based on the estimated second heat flux and the third temperature, and to estimate a body temperature of a user based on the estimated core temperature at the body measurement location and the estimated ambient temperature outside the main body.

The processor may obtain a heat loss from a body reference location to the body measurement location, and may estimate the body temperature by correcting the core temperature at the body measurement location based on the obtained heat loss.

According to another aspect of the present disclosure, there is provided an electronic device including: a memory for storing one or more instructions; a communication interface configured to receive first, second, and third temperatures measured by a plurality of temperature sensors; and a processor configured to estimate a body temperature of a user by executing the one or more instructions, wherein the processor is configured to estimate a first heat flux based on the first temperature and the second temperature, to estimate a core temperature at a body measurement location based on the estimated first heat flux and the first temperature, to estimate a second heat flux based on the second temperature and the third temperature, to estimate an ambient temperature outside a main body based on the estimated second heat flux and the third temperature, and to estimate a body temperature of a user based on the estimated core temperature at the body measurement location and the estimated ambient temperature outside the main body.

### BRIEF DESCRIPTION OF THE DRAWINGS

The above and/or other aspects will be more apparent by describing certain example embodiments, with reference to the accompanying drawings, in which:

FIGS. 1A and 1B are block diagrams illustrating an electronic device according to an embodiment of the present disclosure;

FIGS. 2A and 2B are diagrams illustrating a structure of an electronic device according to an embodiment of the present disclosure;

FIGS. 3A, 3B, and 3C illustrate an example of estimating a blood flow volume based on features of a pulse wave signal according to an embodiment of the present disclosure;

FIG. 4 is a block diagram illustrating an electronic device for estimating body temperature according to another embodiment of the present disclosure;

FIG. 5 is a flowchart illustrating a method of estimating body temperature according to an embodiment of the present disclosure; and

FIGS. 6 to 14 are diagrams illustrating examples of structures of an electronic device; and

FIG. 15 illustrates experimental results of temperature measurements using temperature sensors according to embodiments of the present disclosure.

### DETAILED DESCRIPTION

Example embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the example embodiments. However, it is apparent that the example embodiments can be practiced without those specifically defined matters. Also, well-known functions or constructions are not described in detail since they would obscure the description with unnecessary detail.

Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, all of a, b, and c, or any variations of the aforementioned examples.

It will be understood that, although the terms first, second, etc. may be used herein to describe various elements, these elements should not be limited by these terms. These terms are only used to distinguish one element from another. Any references to singular may include plural unless expressly stated otherwise. In addition, unless explicitly described to the contrary, an expression such as “comprising” or “including” will be understood to imply the inclusion of stated elements but not the exclusion of any other elements. Also, the terms, such as ‘unit’ or ‘module’, etc., should be understood as a unit that performs at least one function or operation and that may be embodied as hardware, software, or a combination thereof.

An electronic device according to various embodiments of the present disclosure which will be described below may include, for example, at least one of a wearable device, a smartphone, a tablet personal computer (PC), a mobile phone, a video phone, an electronic book reader, a desktop computer, a laptop computer, a netbook computer, a workstation, a server, a personal digital assistant (PDA), a portable multimedia player (PMP), an Moving Picture Experts Group Layer-3 Audio (MP3) player, a medical device, and a camera. The wearable device may include at least one of an accessory type wearable device (e.g., wristwatch, ring, bracelet, anklet, necklace, glasses, contact lens, or head mounted device (HMD)), a textile/clothing type wearable device (e.g., electronic clothing), a body-mounted type wearable device (e.g., skin pad or tattoo), and a body implantable type wearable device. However, the wearable device is not limited thereto and may include, for example, various portable medical measuring devices (e.g., antioxidant measuring device, blood glucose monitor, heart rate monitor, blood pressure measuring device, thermometer, etc.), magnetic resonance angiography (MRA), magnetic resonance imaging (MRI), computed tomography (CT), imaging system, ultrasonic system, etc.), and the like. However, the electronic device is not limited to the above devices.

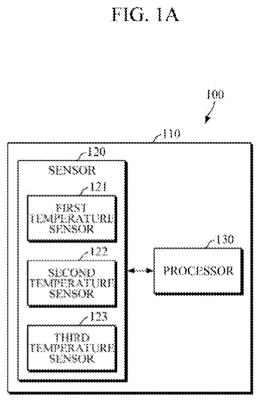

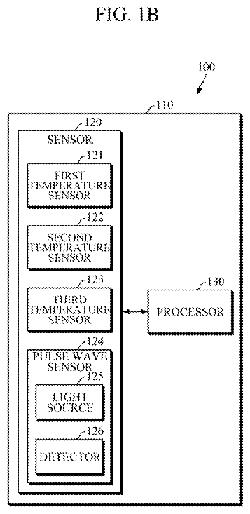

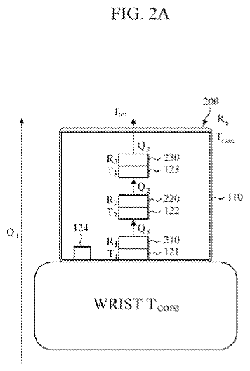

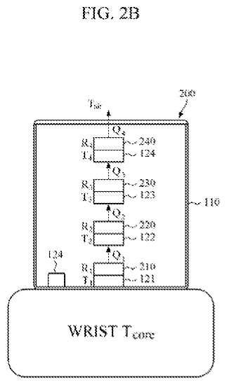

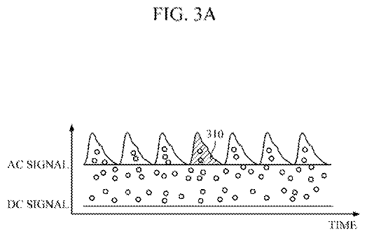

FIGS. 1A and 1B are block diagrams illustrating an electronic device according to an embodiment of the present disclosure. FIGS. 2A and 2B are diagrams illustrating a structure of an electronic device according to an embodiment of the present disclosure.

Referring to FIG. 1A, an electronic device 100 may include a sensor 120 and a processor 130 which are disposed in a main body 110 of the electronic device 100. The sensor 120 may include a plurality of sensors to obtain data for estimating body temperature, and the processor 130 may estimate a user's body temperature by using the data obtained by the sensor 120.

The sensor 120 may include a first temperature sensor 121, a second temperature sensor 122, and a third temperature sensor 123 which may be disposed at different positions in the main body 110. For example, the first temperature sensor 121 may be disposed at a vertical distance (i.e., a thickness direction) of 5 mm or less from a contact surface between the main body 110 and the user's body; and the third temperature sensor 123 may be disposed at a vertical distance of 10 mm or less from the contact surface of the main body 110. In addition, a vertical distance between the first temperature sensor 121 and the second temperature sensor 122 may be 10 mm or less, and a distance between the third temperature sensor 123 and the second temperature sensor 122 may be 50 mm or less.

In an example embodiment, any two or all of the first temperature sensor 121, the second temperature sensor 122, and the third temperature sensor 123 may be disposed in a straight line, and the first temperature sensor 121, the second temperature sensor 122, and the third temperature sensor 123 may be arranged in the main body 110 to be spaced apart from each other. However, the arrangement of the first temperature sensor 121, the second temperature sensor 122, and the third temperature sensor 123 is not limited thereto. For example, the first temperature sensor 121 and the second temperature sensor 122 may be disposed on a rear surface of the main body 110 to be contacted by a wrist, and the third temperature sensor 123 may be disposed on a side surface of the main body 110 to be contacted by a finger. At least one of the first temperature sensor 121, the second temperature sensor 122, and the third temperature sensor 123 may be implemented as a thermistor.

In addition, referring to FIG. 1B, the sensor 120 may further include a pulse wave sensor 124. The pulse wave sensor 124 may be also referred to as a photoplethysmography (PPG) sensor. The pulse wave sensor 124 may measure a pulse wave signal, including a PPG signal, from a user's body and may measure a plurality of pulse wave signals having different wavelengths. In particular, the different wavelengths may include green, blue, red, and infrared wavelengths, and the like.

The pulse wave sensor 124 may include one or more light sources 125 for emitting light of different wavelengths, and one or more detectors 126 for detecting light of different wavelengths scattered or reflected from a skin surface or body tissue such as blood vessels. The light source 125 may include a light emitting diode (LED), a laser diode (LD), a phosphor, etc., but is not limited thereto. In addition, the detector 126 may include a photodiode, a photo transistor (PTr), an image sensor (e.g., complementary metal-oxide semiconductor (CMOS) image sensor), etc., but is not limited thereto. In order to measure two or more pulse wave signals, the pulse wave sensor 124 may include an array of one or more light sources and/or an array of one or more detectors. In particular, the one or more light sources may emit light of different wavelengths, and the respective light sources may be disposed at different distances from the detector.

In an example embodiment, the light source 125 may flash LED lights a preset number of times per second to obtain PPG signals. For example, the light source 125 may use green LED lights to obtain an PPG signal during workouts, and may use red or infrared LED lights to obtain an PPG signal while a software program application for measuring a body temperature is running in the background of the electronic device 100.

Referring to FIG. 2A, in the electronic device 100, in the order of distance from a contact surface between the electronic device 100 and a user, the first temperature sensor 121, the second temperature sensor 122, and the third temperature sensor 123 may be disposed. Specifically, the first temperature sensor 121 may be disposed at a position closest to a contact portion (e.g., wrist, palm of the hand, ankle, auricle, upper arm, etc.) with a user's body, and may measure a first temperature T₁ which represents a surface temperature of the body position. The first temperature T₁ may be also referred to as a skin temperature. The second temperature sensor 122 may be disposed at a position (e.g., upper side of the first temperature sensor 121) spaced apart from the first temperature sensor 121 and may measure a second temperature T₂ inside the main body 110. The third temperature sensor 123 may be disposed at a position (e.g., upper side of the second temperature sensor 122) further away from the first temperature sensor than the second temperature sensor and may measure a third temperature T₃ inside the main body 110. The second temperature T₂ and the third temperature T₃ may be also referred to as a first internal temperature and a second internal temperature of the main body 110, respectively.

In addition, a first thermally conductive material 210 may be disposed between the first temperature sensor 121 and the second temperature sensor 122; a second thermally conductive material 220 may be disposed between the second temperature sensor 122 and the third temperature sensor 123; and a third thermally conductive material 230 may be disposed at an upper end of the third temperature sensor 123.

The first thermally conductive material 210, the second thermally conductive material 220, and/or the third thermally conductive material 230 may be insulators having a thickness of 0.1 mm to 20 mm, and may be materials (e.g., polyurethane foam) having a thermal conductivity of 0.1 W/mK or lower. Further, an air-filled structure may also be provided in which air having a very low thermal conductivity is filled between the first temperature sensor 121 and the second temperature sensor 122, between the second temperature sensor 122 and the third temperature sensor 123, or at an upper portion of the third temperature sensor 123, without using a separate material.

In another embodiment, the first thermally conductive material 210, the second thermally conductive material 220, and the third thermally conductive material 230 may refer to the entire space including air and structures in the electronic device 100. For example, the first thermally conductive material 210 may refer to the entire space including air and/or structures between the first temperature sensor 121 and the second temperature sensor 122, the second thermally conductive material 220 may refer to the entire space including air and/or structures between the second temperature sensor 122 and the third temperature sensor 123, and the third thermally conductive material 230 may refer to the entire space including air and/or structures between the upper portion of the third temperature sensor 123 and upper surface of the main body. In particular, the first thermally conductive material 210, the second thermally conductive material 220, and the third thermally conductive material 230 may have a thermal conductivity of 10 W/mK or lower.

The processor 130 may estimate a core temperature at a measurement location on the body (or body measurement location) and an ambient temperature outside the main body 110 based on data obtained by the plurality of sensors, and may estimate a user's body temperature based on the estimated core temperature at the body measurement location and the ambient temperature outside the main body.

First, the processor 130 may estimate a first heat flux Q₁ based on the first temperature T₁ and the second temperature T₂, and may estimate the core temperature at the body measurement location based on the estimated first heat flux Q₁ and the first temperature T₁.

For example, the processor 130 may estimate the first heat flux Q₁ based on a temperature difference or a temperature gradient between the second temperature T₂ and the first temperature T₁. The temperature gradient may show a change in temperature over a specific distance between the two locations of the first temperature T₁ and the second temperature T₂ (i.e., the locations of two temperature sensors that measure the first temperature T₁ and the second temperature T₂, respectively). Throughout the specification, the term “temperature difference” may include a temperature gradient as well as a difference between two temperatures. Assuming that a flow of heat is a current, a heat transfer property of a material is resistance, and a heat flux is a voltage, the flow of heat may be expressed by an equation according to Bohr's law (V=IR), and a temperature difference (T₁−T₂) in a material may be estimated as the heat flux Q₁. In particular, when the flow of heat from the center of the body measurement location to the surface is in series with the flow of heat from the first temperature T₁ to the second temperature T₂, Equation 1 and 2 below may be derived, and finally, by combining the estimated first heat flux Q₁ with the first temperature T₁ which is a surface temperature to be measured at the body measurement location, the core temperature at the body measurement location may be estimated and may be represented by the following Equation 3.

$$\frac{{T}_{c o r e} - {T}_{1}}{{R}_{s k i n}} = \frac{{T}_{1} - {T}_{2}}{{R}_{1}} \qquad [ Equation 1 ]$$

$$\frac{{T}_{c o r e} - {T}_{1}}{{T}_{1} - {T}_{2}} = \frac{{R}_{s k i n}}{{R}_{1}} = \frac{1}{ε} \qquad [ Equation 2 ]$$

$${T}_{c o r e} = {T}_{1} + \frac{{T}_{1} - {T}_{2}}{ε} \qquad [ Equation 3 ]$$

Herein, T_{core} denotes the core temperature at the body measurement location, Rₛₖᵢₙ denotes a predetermined skin thermal resistance value, R₁ denotes a resistance value of the first thermally conductive material 210, and ε denotes a ratio of the Rₛₖᵢₙ and R₁. At this time, may be stored as a predetermined value, for example, in a storage.

The processor 130 may estimate a skin blood flow volume based on the pulse wave signal (e.g., a PPG signal) measured by the pulse wave sensor 124, and may estimate the core temperature at the body measurement location by further using the estimated skin blood flow volume in addition to the first heat flux Q₁ and the first temperature T₁. By estimating the skin blood flow volume during estimation of the core temperature at the body measurement location, it is possible to correct a change in skin thermal conductivity due to a blood flow change, and thus the core temperature at the body measurement location may be estimated accurately.

For example, the processor 130 may estimate the first heat flux Q₁ based on the temperature difference T₁−T₂ between the first temperature T₁ and the second temperature T₂, and may estimate the core temperature T_{core} at the body measurement location by combining a ratio between the estimated first heat flux Q₁ and the skin blood flow volume with the first temperature T₁, which may be represented by the following Equation 4.

$${T}_{c o r e} = {T}_{1} + \frac{{T}_{1} - {T}_{2}}{ε + α {F}_{s}} \qquad [ Equation 4 ]$$

Herein, Fₛ denotes the skin blood flow volume, and ε and α denote predetermined coefficients.

In this case, the processor 130 may extract a feature from the pulse wave signal, and may obtain the skin blood flow volume from the extracted feature by using a model that defines a correlation between the feature and the skin blood flow volume.

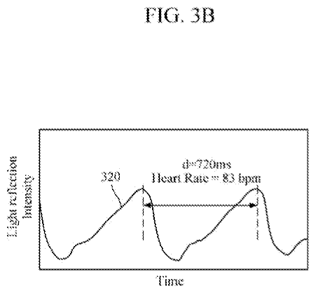

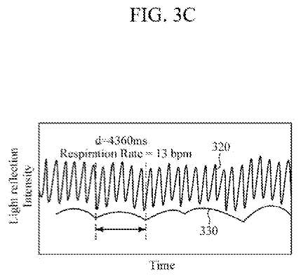

FIGS. 3A, 3B, and 3C illustrate an example of estimating a blood flow volume based on features of a pulse wave signal according to an embodiment of the present disclosure.

Referring to FIG. 3A, a pulse wave signal includes direct current (DC) and alternating current (AC) signals, in which the DC signal is a constant signal (with a margin) that is reflected from a bone, a muscle, etc., and the AC signal is a signal that changes as blood pumped out of the heart flows into the artery. Referring to FIG. 3A, an upper portion of the graph is the AC signal that changes over time, and a lower portion is the DC signal which is a constant signal. The AC signal may indicate whether an arterial blood volume increases or decreases, thereby indirectly reflecting a change in blood flow volume. Accordingly, the processor 130 may extract an area under curve (AUC) 310 of the AC component of the pulse wave signal, as the feature of the pulse wave signal, and may estimate a blood flow volume by using the extracted AUC. The AUC 310 of the AC component of the pulse wave signal may refer to an area between an upper envelope of the pulse wave signal and a baseline that connects local minimum values of the AC component of the pulse wave signal. However, the method of estimating the blood flow volume is not limited to using the AUC, but may include using other AC features, such as a maximum value, a minimum value, a mean, and/or a slope of the AC signal within a predetermined time range may also be used. In particular, by applying the correlation between the AUC and the skin blood flow volume to a predefined blood flow volume estimation model, the skin blood flow volume may be obtained from the extracted AUC.

Referring to FIGS. 3B and 3C, the processor 130 may extract a first AC feature signal 320 and a second AC feature signal 330 from the AC signal via one or more band-pass filters that selectively pass a preset frequency range of the AC signal. Each of the first AC feature signal and the second AC feature signal may be expressed in a time and a light intensity domain to show a change in light intensity over time. The first AC feature signal may have a shorter period that the second AC feature signal, and may represent a heart rate. The second AC feature signal may have a longer period than the first AC feature signal, and may represent a respiration rate.

In addition, the processor 130 may extract, as features of the pulse wave signal for estimating a blood flow volume, a time associated with a propagation wave component of the pulse wave signal and an amplitude corresponding to the time and a time associated with a reflection wave component of the pulse wave signal and an amplitude corresponding to the time. In particular, the processor 130 may extract characteristic points associated with the propagation wave component and the reflection wave component based on a second derivative signal of the pulse wave signal. For example, the processor 130 may extract a time position at a first local minimum point of the second derivative signal as the time associated with the propagation wave component, and may extract time positions at each of second and third local minimum points as the time associated with the reflection wave component. Further, the processor 130 may extract, as characteristic points, a time and/or an amplitude at a predetermined point in a predetermined region of the pulse wave signal, for example, a time and/or an amplitude at a point where the amplitude is maximum, a time at an internally dividing point between the time at the point where the amplitude is maximum and the time associated with the propagation wave component, and/or an amplitude corresponding to the time point. In this case, the internally dividing point may be a middle point between two time points or a point obtained by internally dividing the two time points in a predetermined ratio.

Referring back to FIG. 2A, the processor 130 may estimate a second heat flux Q₂ based on the second temperature T₂ and the third temperature T₃, and may estimate an ambient temperature outside the main body 110 based on the estimated second heat flux Q₂ and the third temperature T₃.

For example, the processor 130 may estimate the second heat flux Q₂ based on a time difference T₂−T₃ between the second temperature T₂ and the third temperature T₃, and may estimate the ambient temperature outside the main body 110 by combining the estimated second heat flux Q₂ with the third temperature T₃.

First, the processor 130 may estimate the second heat flux Q₂ based on the time difference T₂−T₃ between the second temperature T₂ and the third temperature T₃. For example, assuming that heat transfer from the surface of the body measurement location to the top of the main body 110 occurs in a series circuit and the heat flux is Q_{T}, a temperature difference between the first temperature T₁ and the second temperature T₂, a temperature difference between the second temperature T₂ and the third temperature T₃, a temperature difference between the third temperature T₃ and a temperature T_{case} of the upper surface of the main body, and a temperature difference between the temperature T_{case} of the upper surface of the main body and the ambient temperature Tₐᵢᵣ outside the main body may be estimated based on the same heat flux Q_{T}. In this case, as for the flow of heat at the inner portion and the top of the main body 110, the following Equation 5 may be derived based on Bohr's law (V=IR).

$$\frac{{T}_{c a s e} - {T}_{a i r}}{{R}_{s}} = \frac{{T}_{3} - {T}_{c a s e}}{{R}_{3}} = \frac{{T}_{2} - {T}_{3}}{{R}_{2}} \qquad [ Equation 5 ]$$

Herein, R₂ denotes a resistance value of the second thermally conductive material 220, R₃ denotes a resistance value of the third thermally conductive material 230, and Rₛ denotes a resistance value of the upper surface of the main body. In this case, assuming that heat leaks from the side of the main body 110, an equation such as the above Equation 5 may be degraded, and the respective terms in Equation 5 are in a proportional relationship and may be multiplied by predetermined coefficients.

The temperature T_{case} of the upper surface of the main body and the ambient temperature Tₐᵢᵣ outside the main body may be represented by the following Equations 6 and 7.

$${T}_{c a s e} = {T}_{3} - \frac{{R}_{3}}{{R}_{2}} ( {T}_{2} - {T}_{3} ) \qquad [ Equation 6 ]$$

$${T}_{a i r} = {T}_{case} + \frac{{R}_{s}}{{R}_{3}} ( {T}_{c a s e} - {T}_{3} ) \qquad [ Equation 7 ]$$

Then, by substituting Equation 6 into Equation 7, the ambient temperature Tₐᵢᵣ outside the main body may be represented by the following Equation 8.

$${T}_{a i r} = {T}_{3} \frac{{R}_{3} + {R}_{s}}{{R}_{2}} ( {T}_{2} - {T}_{3} ) = {T}_{3} - β ( {T}_{2} - {T}_{3} ) \qquad [ Equation 8 ]$$

Herein, β denotes a correction coefficient and thermal conductivity according to physical properties. The processor 130 may calculate the correction coefficient for correcting the second heat flux Q₂ based on the resistance value R₂ of the second thermally conductive material 220, and the resistance value R₃ of the third thermally conductive material 230, and the resistance value Rₛ of the upper surface 200 of the main body 110. For example, the processor 130 may calculate the correction coefficient based on a ratio between the resistance value R₂ of the second thermally conductive material 220 and a sum of the resistance value R₃ of the third thermally conductive material 210 and the resistance value Rₛ of the upper surface 200 of the main body 110. The calculated correction coefficient may be prestored in a storage of the electronic device 100.

That is, as shown in the above Equation 8, the processor 130 may measure the ambient temperature Tₐᵢᵣ outside the main body by combining the third temperature T₃ with a result obtained by applying the correction coefficient β to the heat flux Q₂ which is estimated based on the difference T₂−T₃ between the second temperature and the third temperature.

When ambient temperature around an electronic device is measured using a temperature sensor of the electronic device, it may be difficult to measure the temperature accurately due to body heat of a user being in contact with the electronic device. The above embodiment of the present disclosure provides a method of estimating a heat flux from a user by using a plurality of temperature sensors and estimating the ambient temperature based on the estimated heat flux, in which by offsetting the effect of body heat, the accuracy of estimating the ambient temperature around the electronic device may be improved.

FIG. 2B is a diagram illustrating a structure of an electronic device according to another embodiment of the present disclosure.

Referring to FIG. 2B, the main body 110 of the electronic device 100 may further include a fourth temperature sensor 124, in addition to the first temperature sensor 121, the second temperature sensor 122, and the third temperature sensor 123. The fourth temperature sensor 124 may be disposed at a position (e.g., upper side of the third temperature sensor 123) further away from the first temperature sensor 121 than from the third temperature sensor 123 and may measure a fourth temperature T₄ inside the main body 110. In addition, the first thermally conductive material 210 may be disposed between the first temperature sensor 121 and the second temperature sensor 122; the second thermally conductive material 220 may be disposed between the second temperature sensor 122 and the third temperature sensor 123; the third thermally conductive material 230 may be disposed between the third temperature sensor 123 and the fourth temperature sensor 124; and a fourth thermally conductive material 240 may be disposed at an upper end of the fourth temperature sensor 124. In particular, at least one of the first temperature sensor 121, the second temperature sensor 122, the third temperature sensor 123, and the fourth temperature sensor 124 may be a thermistor, and at least one of the first thermally conductive material 210, the second thermally conductive material 220, the third thermally conductive material 230, and the fourth thermally conductive material 240 may have air.

In particular, the third temperature sensor 123, for example, may be attached to a structure in the main body 10, such that the third temperature sensor 123 may further measure temperature of heat generated from the structure.

By measuring the second temperature using the second temperature sensor 122, the third temperature using the third temperature sensor 123, and the fourth temperature using the fourth temperature sensor 124, the processor 130 may measure the ambient temperature outside the main body based on the measured temperatures. For example, the processor 130 may estimate the second heat flux Q₂ based on a temperature difference T₂−T₃ between the second temperature and the third temperature, estimate a third heat flux Q₃ based on a temperature difference T₃−T₄ between the third temperature and the fourth temperature, and may measure the ambient temperature outside the main body based on the estimated second heat flux Q₂ and third heat flux Q₃, and the fourth temperature.

First, when heat is generated from the structure, to which the third temperature sensor 123 is attached, in the main body 310, a heat flux flowing from the wrist toward the top of the main body 110 through the third temperature sensor 123 is not constant, such that there is a difference between the second heat flux Q₂ and the third heat flux Q₃. For example, due to the heat generated from the structure, the second heat flux Q₂ may relatively increase, and the third heat flux Q₃ may relatively decrease, which may be expressed by the following Equation 9 according to Bohr's law.

$$\frac{{T}_{2} - {T}_{3}}{{R}_{2}} > \frac{{T}_{3} - {T}_{4}}{{R}_{3}} \qquad [ Equation 9 ]$$

In particular, by reflecting the heat Tᵢₙ, which is generated from the structure, in Equation 9, a flow of heat from the wrist may be expressed by the following Equation 10, including a fourth heat flux Q₄ estimated based on a temperature difference T₄−Tₐᵢᵣ between the fourth temperature and the temperature at the top of the main body 110.

$$\frac{{T}_{2} - {T}_{3} - {T}_{i n}}{{R}_{2}} = \frac{{T}_{3} - {T}_{4} + {T}_{i n}}{{R}_{3}} = \frac{{T}_{4} - {T}_{a i r}}{{R}_{4}} \qquad [ Equation 10 ]$$

Herein, R₂ denotes the resistance value of the second thermally conductive material 220, R₃ denotes the resistance value of the third thermally conductive material 230, and R₄ denotes the resistance value of the fourth thermally conductive material 240.

Assuming that heat leaks from the side of the main body 110, an equation such as the above Equation 10 may be degraded, and the respective terms in the equation are in a proportional relationship and may be multiplied by predetermined coefficients.

By using the above Equation 10, the heat Tᵢₙ generated from the structure and the air temperature Tₐᵢᵣ at the top of the main body may be expressed by the following Equations 11 and 12, respectively.

$${T}_{i n} = \frac{{R}_{3} ( {T}_{2} - {T}_{3} ) - {R}_{2} ( {T}_{3} - {T}_{4} )}{{R}_{2} + {R}_{3}} \qquad [ Equation 11 ]$$

$${T}_{a i r} = {T}_{4} - \frac{{R}_{4}}{{R}_{3}} ( {T}_{3} - {T}_{4} + {T}_{i n} ) \qquad [ Equation 12 ]$$

Then, by substituting Equation 11 into Equation 12, the air temperature Tₐᵢᵣ at the top of the main body may be expressed by the following Equation 13.

$${T}_{a i r} = {T}_{4} - \frac{{R}_{4}}{{R}_{3}} ( {T}_{3} - {T}_{4} + \frac{{R}_{3} ( {T}_{2} - {T}_{3} ) - {R}_{2} ( {T}_{3} - {T}_{4} )}{{R}_{2} + {R}_{3}} ) \qquad [ Equation 13 ]$$

Based on the above Equation 11, the processor 130 may estimate the temperature Tᵢₙ of the heat generated from the structure in the main body based on a difference between the second heat flux Q₂, estimated based on the difference T₂−T₃ between the second temperature and the third temperature, and the third heat flux Q₃ estimated based on the difference T₃−T₄ between the third temperature and the fourth temperature. In addition, based on the above Equation 11, the processor 130 may reflect the temperature Tᵢₙ increased due to the heat generated from the structure in the main body, and may measure the ambient temperature outside the main body based on the resistance value R₂ of the second thermally conductive material, and the resistance value R₃ of the third thermally conductive material, the resistance value R₄ of the fourth thermally conductive material, the estimated second heat flux Q₂ and third heat flux Q₃, and the fourth temperature T₄. In this case, the resistance value R₂ of the second thermally conductive material, the resistance value R₃ of the third thermally conductive material, and the resistance value R₄ of the fourth thermally conductive material may be prestored in the storage.

When an electronic device is used, heat may also be generated from structures included in a main body of the electronic device. According to the above embodiment of the present disclosure, not only the effect of a user's body heat but also the effect of heat generated from the structures may be offset, thereby improving the accuracy of estimating the ambient temperature outside the electronic device.

Then, the processor 130 may estimate a user's body temperature based on the estimated core temperature at the body measurement location and the ambient temperature outside the main body.

For example, by obtaining a heat loss from a body reference location to the body measurement location, and by correcting the core temperature T_{core} at the body measurement location based on the obtained heat loss, the processor 130 may estimate body temperature T_{body}, which may be represented by the following Equation 14. In this case, the body reference location may be, for example, a core part of the body, and the body measurement location may be, for example, wrist, ankle, auricle, palm of the hand, upper arm, etc., which are peripheral parts of the body disposed away from the core part. However, the body reference location and the body measurement location are not limited thereto.

T_{body}=T_{core}+Tₗₒₛₛ [Equation 14]

Herein, Tₗₒₛₛ denotes the heat loss from the body reference location to the body measurement location, and may be, for example, a heat loss from the core part to the wrist.

The heat loss may be changed by outside air temperature, and the change occurs by conduction, convection, and radiation. The heat loss occurring by conduction and convection is proportional to a temperature difference and/or a temperature gradient between skin temperature and outside air temperature, and the heat loss occurring by radiation is proportional to the fourth power of a temperature difference and/or a temperature gradient between the skin temperature and the outside air temperature. During estimation of the body temperature, the heat loss may be estimated by using the electronic device based on the relationship.

For example, the processor 130 may estimate the heat loss Tₗₒₛₛ based on a temperature difference and/or a temperature gradient between the first temperature T₁ and the ambient temperature Tₐᵢᵣ outside the main body, which may be represented by the following Equation 15.

Tₗₒₛₛ=γ(T₁−Tₐᵢᵣ)+δ(T₁⁴−Tₐᵢᵣ⁴) [Equation 15]

Herein, Γ and δ are predetermined heat loss coefficients, and may be prestored in the storage of the electronic device 100.

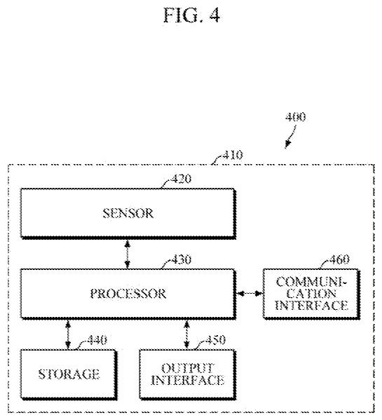

FIG. 4 is a block diagram illustrating an electronic device for estimating body temperature according to another embodiment of the present disclosure.

Referring to FIG. 4, an electronic device 400 may include a sensor 420, a processor 430, a storage 440, an output interface 450, and a communication interface 460 which are included in a main body 410. In this case, the sensor 420 and the processor 430 are the same as those of the sensor 120 and the processor 130 of FIGS. 1A and 1B, such that a detailed description thereof will be omitted.

The storage 440 may store information related to estimating body temperature. For example, the storage 440 may store temperature data and pulse wave signals obtained by the sensor 420, resistance values of thermally conductive materials, correction coefficients, heat loss coefficients, and processing results of the processor 430, for example, heat flux, estimated core temperature values at the body measurement location, estimated ambient temperature values outside the main body, and the like.

The storage 440 may include at least one storage medium of a flash memory type memory, a hard disk type memory, a multimedia card micro type memory, a card type memory (e.g., an SD memory, an XD memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like, but is not limited thereto.

The output interface 450 may provide processing results of the processor 430 for a user. For example, the output interface 450 may display an air or ambient temperature value and/or a body temperature value estimated by the processor 430 on a display. In this case, the output interface 450 may provide the user with information by changing color, line thickness, etc., so that the user may easily recognize the estimated body temperature value. The output interface 450 may also provide information on a continuous body temperature change over time. Further, the output interface 450 may output at least one of the first temperature, the second temperature, the third temperature, the core temperature at the body measurement location, the ambient temperature outside the main body, body temperature, and guidance information related to body temperature to the display. In this case, along with or without the visual display, the output interface 450 may provide the user with body temperature information in a non-visual manner by voice, vibrations, tactile sensation, and the like using an audio output module, such as a speaker and the like, or a haptic module.

The communication interface 460 may communicate with an external device to transmit and receive various data related to estimating body temperature. The external device may include an information processing device, such as a smartphone, a tablet PC, a desktop computer, a laptop computer, and the like. For example, the communication interface 460 may transmit a body temperature measurement result to the external device, such as a smartphone and the like, and a user may monitor the body temperature over time by using, e.g., the smartphone.

The communication interface 460 may communicate with the external device by using various wired and wireless communication techniques including Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, WIFI communication, Radio Frequency Identification (RFID) communication, 3G, 4G, and 5G communications, and the like. However, the communication techniques are not limited thereto.

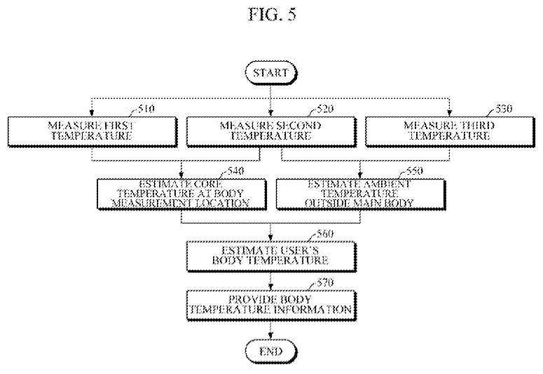

FIG. 5 is a flowchart illustrating a method of estimating body temperature according to an embodiment of the present disclosure.

The method of FIG. 5 is an example of a method of estimating body temperature performed by the electronic devices 100 and 400 according to the embodiments of FIGS. 1 and 4, which are described in detail above, and thus will be briefly described below in order to avoid redundancy.

Referring to FIG. 5, the electronic device may measure a first temperature from the surface of a body measurement location by using the first temperature sensor in the main body in operation 510, measure a second temperature inside the main body by using the second temperature sensor spaced apart from the first temperature sensor in operation 520, and measure a third temperature inside the main body by using the third temperature sensor disposed further away from the first temperature sensor than the second temperature sensor in operation 530.

Then, the electronic device may estimate a first heat flux based on the first temperature and the second temperature, and estimate a core temperature at the body measurement location based on the estimated first heat flux and the first temperature in operation 540. In this case, the electronic device may estimate the first heat flux based on a temperature difference and/or or a temperature gradient between the first temperature and the second temperature. Further, the electronic device may estimate a first skin blood flow volume based on a pulse wave signal (e.g., a PPG signal) measured by the pulse wave sensor, and may also estimate a core temperature at the body measurement location based on the first heat flux, the first temperature, and the estimated skin blood flow volume. Additionally, the electronic device may estimate the first skin blood flow volume based on either one or a combination of a PPG signal and an ECG signal. For example, the electronic device may estimate the core temperature at the body measurement location by combining a ratio between the estimated first heat flux and the skin blood flow volume with the first temperature.

Subsequently, the electronic device may estimate a second heat flux based on the measured second temperature and third temperature, and may estimate an ambient temperature outside the main body based on the estimated second heat flux and the third temperature in operation 550. For example, the electronic device may estimate the second heat flux based on a temperature difference and/or or a temperature gradient between the second temperature and the third temperature, and may estimate the ambient temperature outside the main body by combining the estimated second heat flux and the third temperature. In this case, the electronic device may correct the second heat flux based on a resistance value of a thermally conductive material disposed between the second temperature sensor and the third temperature sensor, a resistance value of a thermally conductive material disposed between the third temperature sensor and the surface of the main body, and a resistance value of the surface of the main body.

Next, the electronic device may estimate a user's body temperature based on the estimated core temperature at the body measurement location and the ambient temperature outside the main body in operation 560. For example, the electronic device may obtain a heat loss from a body reference location to the body measurement location, and may estimate the body temperature by correcting the core temperature at the body measurement location based on the obtained heat loss. In particular, the electronic device may obtain the heat loss based on a temperature difference and/or a temperature gradient between the first temperature and the ambient temperature outside the main body.

Then, the electronic device may provide the user with the estimated body temperature information through the output interface in operation 570. In this case, the body temperature information may include not only the user's estimated body temperature, but also continuous body temperature information that has been continuously measured over time, the first temperature, the second temperature, the third temperature, the core temperature at the body measurement location, the ambient temperature outside the main body, the guidance information related to body temperature, and the like.

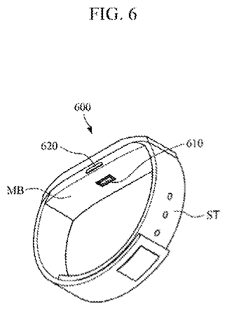

FIGS. 6 to 14 are diagrams illustrating examples of structures of an electronic device.

Referring to FIG. 6, the electronic device may be implemented as a smart watch-type wearable device 600 which includes a main body MB and a wrist strap ST.

The main body MB may be formed in various shapes. A battery may be embedded in the main body MB and/or the strap ST to supply power to various components of the wearable device. The strap ST may be connected to both ends of the main body to allow the main body to be worn on a user's wrist, and may be flexible so as to be wrapped around the user's wrist. The strap ST may be composed of a first strap and a second strap which are separated from each other. One ends of the first strap and the second strap are connected to both sides of the main body MB, and the other ends thereof may be connected to each other via a fastening means. In particular, the fastening means may be formed as magnetic fastening, Velcro fastening, pin fastening, etc., but is not limited thereto. Further, the strap ST is not limited thereto, and may be integrally formed as a non-detachable band.

The main body MB may include a sensor 610, a processor, an output interface, a storage, and a communication interface. However, depending on the size and shape of a form factor and the like, some of the output device, the storage, and the communication interface may be omitted.

The sensor 610 may include a first temperature sensor for measuring a first temperature, a second temperature sensor for measuring a second temperature, and a third temperature sensor for measuring a third temperature, in which the first, second, and third temperature sensors may be disposed at different distances from a body contact surface. In particular, the first temperature may be a surface temperature at a body measurement location, and the second temperature and the third temperature may be different temperatures inside the main body which are measured by the second and third temperature sensors spaced apart from each other. In addition, the sensor 610 may further include a pulse wave sensor (e.g., a PPG sensor) including a light source and a detector and configured to measure a user's pulse wave signal and an ECG (electrocardiography) sensor. One of the electrodes of the ECG sensor may be disposed on a side surface of the main body MB, for example, at a manipulator 620.

In particular, the sensor 610 may be disposed on a rear surface of the main body MB to obtain data for measuring body temperature when the main body MB is worn on the user's wrist.

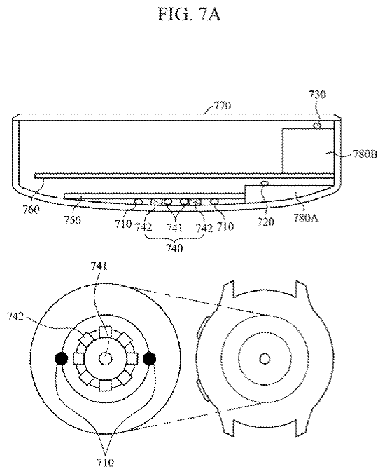

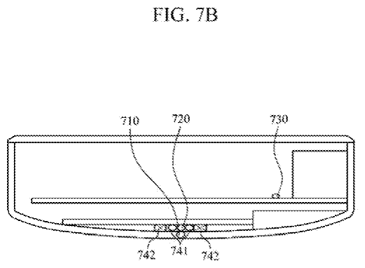

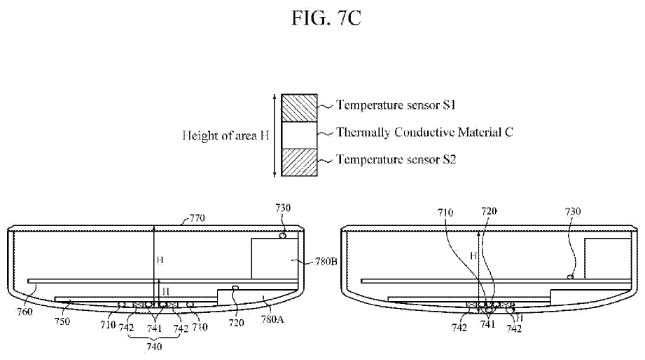

FIGS. 7A and 7B are diagrams illustrating the arrangement of sensors in a wearable device.

Referring to FIGS. 7A and 7B, a pulse wave sensor 740, including a plurality of light sources 741 and detectors 742, and a first temperature sensor 710 may be disposed on a contact surface of the wearable device, wherein the contact surface is to be in contact with a wrist (or another body part) of a user when the wearable device is worn by the user. The first temperature sensor 710 and the pulse wave sensor 740 may be electrically connected to a sensor circuit board 750. In addition, a second temperature sensor 720 may be disposed directly on top of the first temperature sensor 710 or disposed above the first temperature sensor 710, and the third temperature sensor 730 may be disposed at a position closer to a front surface of the main body MB than the second temperature sensor 720, so as to easily measure an ambient temperature outside the main body. In particular, referring to FIG. 7A, the second temperature sensor 720 and the third temperature sensor 730 may be mounted on a first supporting structure 780A, and a second supporting structure 780B, respectively. The second temperature sensor 720 may be disposed anywhere between the first temperature sensor 720 and a main circuit board 760 in a vertical direction (e.g., a thickness direction) of the wearable device. The third temperature 730 may be disposed anywhere between the mainboard 760 and a display panel 770 in the vertical direction. Any two or all of the first temperature sensor 710, the second temperature sensor 720, and the third temperature sensor 730 may be arranged in a straight light in the vertical direction of the wearable device.

FIG. 7C illustrates an arrangement and sizes of temperature sensors and a thermally conductive material. The temperature sensors S1 and S2 may correspond to any two temperature sensors among the first temperature sensor 121, the second temperature sensor 122, the third temperature sensor 123, and the fourth temperature sensor 124 illustrated in FIGS. 2A and 2B. The thermally conductive material C may correspond to any thermally conductive material (e.g., a thermally conductive material 210, 220, 230, or 240) that is provided between the two temperature sensors S1 and S2. The thermally conductive material C may be disposed directly on either one or both of the temperature sensors S1 and S2. For example, there may be no space between the thermally conductive material C and each of the temperature sensors S1 and S2, or alternatively, there may be a space or a gap between the thermally conductive material C and one of the temperature sensors S1 and S2 while being in contact with the other one of the temperature sensors S1 and S2.

The two temperature sensors S1 and S2 and the thermally conductive material C may be provided in an area between the contact surface and the display panel 770. In order to implement a body temperature measuring device as a smart watch, there may be restrictions on a height of the temperature sensors S1 and S2 and a height of the thermally conductive material C (or a distance between the temperature sensors S1 and S2), since the area of the smart watch that can accommodate the temperature sensors S1 and S2 is small. For example, the height of the area of the smart watch that can accommodate the temperature sensors S1 and S2 may be in a range from 1 mm to 1.5 mm. Given the limited height of the area in the smart watch, the height of the thermally conductive material C may decrease as the height of the temperature sensors S1 and S2 increases, while a certain distance between the two temperature sensors S1 and S2 is required to obtain a minimum temperature difference (e.g., 0.3° C.) between the two temperature sensors S1 and S2 and thereby to estimate a body temperature based on the temperature difference. Since the temperature sensors S1 and S2 may have some error rate (e.g., ±0.1° C.), it may be difficult to reliably measure the temperature difference between the two temperature sensors S1 and S2 when a target temperature difference between the two temperature sensors S1 and S2 is set to be less than 0.3° C. Based on such understanding, a minimum target temperature difference between the two temperature sensors S1 and S2 may be set to 0.3° C., and a heat transfer simulation has been conducted by changing the height of the temperature sensors S1 and S2, and the height of the thermally conductive material C for each of a plurality of area heights H, as shown below in Table 1.

TABLE 1

| | | Height of Thermally | |
|---|---|---|---|
| | | Conductive Material | |
| | | (e.g., insulator | |
| Area | Sensor | and/or air between | Temperature |
| height H | Height | temperature sensors) | Difference |

| | | | | |
|---|---|---|---|---|
| 1 | mm | 0.1 | 0.8 | 0.648 |
| | | 0.2 | 0.6 | 0.486 |
| | | 0.3 | 0.4 | 0.324 |
| | | 0.4 | 0.2 | 0.162 |
| 1.1 | mm | 0.1 | 0.9 | 0.730 |
| | | 0.2 | 0.7 | 0.567 |
| | | 0.3 | 0.5 | 0.405 |
| | | 0.4 | 0.3 | 0.243 |
| 1.2 | mm | 0.1 | 1 | 0.811 |
| | | 0.2 | 0.8 | 0.648 |
| | | 0.3 | 0.6 | 0.486 |
| | | 0.4 | 0.4 | 0.324 |
| | | 0.5 | 0.2 | 0.162 |
| 1.3 | mm | 0.1 | 1.1 | 0.892 |
| | | 0.2 | 0.9 | 0.730 |
| | | 0.3 | 0.7 | 0.567 |
| | | 0.4 | 0.5 | 0.405 |
| | | 0.5 | 0.3 | 0.243 |
| 1.4 | mm | 0.1 | 1.2 | 0.973 |
| | | 0.2 | 1 | 0.811 |
| | | 0.3 | 0.8 | 0.648 |
| | | 0.4 | 0.6 | 0.486 |
| | | 0.5 | 0.4 | 0.324 |
| | | 0.6 | 0.2 | 0.162 |
| 1.5 | mm | 0.1 | 1.3 | 1.054 |
| | | 0.2 | 1.1 | 0.892 |
| | | 0.3 | 0.9 | 0.730 |
| | | 0.4 | 0.7 | 0.567 |
| | | 0.5 | 0.5 | 0.405 |
| | | 0.6 | 0.3 | 0.243 |

Referring to Table 1 above, when a target temperature difference between the two temperature sensors S1 and S2 is greater than or equal to 0.3° C., the height of each of the temperature sensors S1 and S2 may be set to have a minimum height of 0.3 mm (i.e., 0.3 mm or greater, and preferably from 0.3 mm to 0.5 mm), and the height of the thermally conductive material C may be set to a minimum height of 0.4 mm or (i.e., 0.4 mm or greater, and preferably from 0.4 mm to 1.3 mm).

Referring to FIGS. 7A, 7B, and 7C, the first temperature sensor 170 may be disposed as close as possible to the contact surface, and the third temperature sensor 730 may be disposed as close as possible to the display panel 770 to provide a relatively accurate temperature estimation.

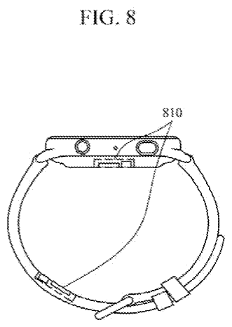

Further, referring to FIG. 8, sensors 810 may be disposed not only on the rear surface of the main body MB but also on the wrist strap ST, to obtain data.

Referring back to FIG. 6, a manipulator 620 may be formed on a side surface of the main body MB, as illustrated herein. The manipulator 620 may receive a user's command and may transmit the received command to the processor. In addition, the manipulator 620 may have a power button to turn on/off the wearable device 600.

The processor mounted in the main body MB may be electrically connected to various components including the sensor 610. The processor may estimate a user's body temperature by using data obtained by the plurality of sensors 610. For example, while the main body MB is worn, the processor may estimate a first heat flux based on the first temperature and the second temperature, estimate a core temperature at the body measurement location based on the estimated first heat flux and the first temperature, estimate a second heat flux based on the second temperature and the third temperature, estimate an ambient temperature outside the main body based on the estimated second heat flux and the third temperature, and estimate the user's body temperature based on the estimated core temperature at the body measurement location and the estimated ambient temperature outside the main body. In particular, the processor may obtain a heat loss from a body reference location (e.g., core part) to the body measurement location (e.g., wrist), and may obtain the body temperature by correcting the core temperature at the body measurement location based on the obtained heat loss.

A display may be provided on the front surface of the main body MB and may display various application screens, including body temperature information, time information, received message information, and the like. For example, an estimated body temperature value may be displayed on the display. In particular, if the estimated body temperature value falls outside a normal range, the processor may provide a user with warning information by changing color, line thickness, etc., or displaying an abnormal value along with the normal range, so that the user may easily recognize the abnormal value. Further, in response to a user's request, the processor may display not only the current estimated body temperature value, but also continuous estimated body temperature values over time and may provide the values to the user. In addition, the processor may display, on the display, a variation in body temperature, e.g., a body temperature change during a day in graph form, and information as to whether the user has a deep sleep based on the body temperature change. The information that may be displayed on the display may include not only the body temperature, but also the measured first, second, and third temperatures, the core temperature at the body measurement location, the ambient temperature outside the main body, guidance information related to body temperature, etc., but is not limited thereto.

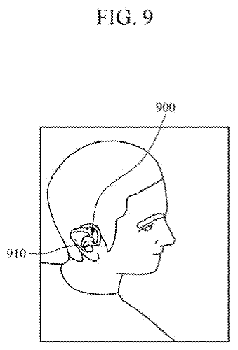

Referring to FIG. 9, the electronic device may be implemented as an ear-wearable device 900.

The ear-wearable device 900 may include a main body and an ear strap. A user may wear the ear-wearable device 900 by hanging the ear strap on the user's auricle. The ear strap may be omitted depending on the shape of ear-wearable device 900. The main body may be inserted into the external auditory meatus. A sensor 910 may be mounted in the main body. The ear-wearable device 900 may provide the user with a body temperature estimation result as sound, or may transmit the estimation result to an external device, e.g., a mobile device, a tablet PC, a personal computer, etc., through a communication module provided in the main body.

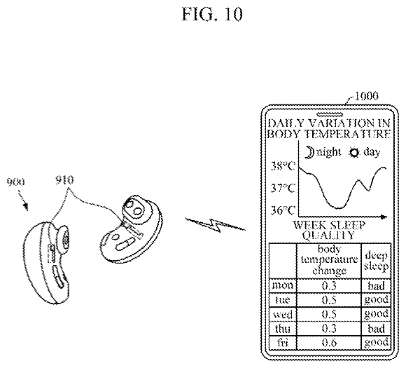

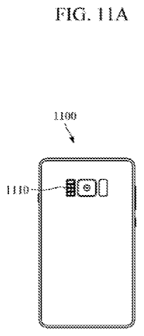

Referring to FIG. 10, the electronic device may be implemented by a combination of an ear-wearable device and a mobile device such as a smartphone. However, this is merely an example, and various combinations of electronic devices may be provided. For example, a processor for estimating body temperature may be mounted in a main body of a mobile device 1000. Upon receiving a request for measuring body temperature, the processor of the mobile device 1000 may control a communication interface to communicate with a communication module mounted in the main body of the wearable device 900, to obtain data by using the sensor 910. Further, upon receiving data, such as the first temperature, the second temperature, the third temperature, and/or the pulse wave signal, etc., from the wearable device 900, the processor may estimate body temperature, and may output an estimation result and information related to body temperature to the display of the mobile device 1000 through an output interface as illustrated herein. For example, in response to a user's request, the processor may display not only the current estimated body temperature value, but also continuous estimated body temperature values over time on the display to provide the values to the user. In addition, the processor may display, on the display, a variation in body temperature, e.g., a body temperature change during a day in graph form, and information as to whether the user has a deep sleep based on the body temperature change.

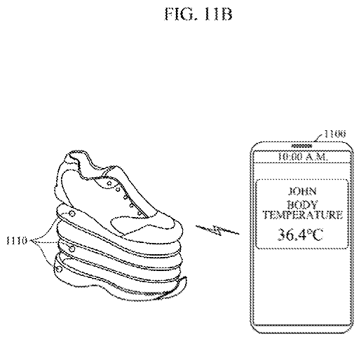

Referring to FIG. 11A, the electronic device may be implemented as a mobile device 1100 such as a smartphone.

The mobile device 1100 may include a housing and a display panel. The housing may form an exterior of the mobile device 1100. The housing has a first surface, on which a display panel and a cover glass may be disposed sequentially, and the display panel may be exposed to the outside through the cover glass. A sensor 1110, a camera module and/or an infrared sensor, and the like may be disposed on a second surface of the housing.

For example, a plurality of sensors for obtaining data from a user may be disposed on a rear surface of the mobile device 1100, and a fingerprint sensor disposed on the front surface of the mobile device 1100, a power button or a volume button disposed on a side surface the mobile device 1100, a sensor disposed on other positions of the front and rear surfaces of the mobile device 1100, and the like may be provided to measure body temperature.

In addition, when a user transmits a request for estimating body temperature by executing an application and the like installed in the mobile device 1100, the mobile device 1100 may obtain data by using the sensor 1110, and may estimate the body temperature and may provide the estimated value as images and/or sounds to the user by using the processor in the mobile device 1100.

Furthermore, the sensor 1110 may be disposed not only inside the mobile device 1100 but also outside the mobile device 1100 at a position close to a body measurement location.

Referring to FIG. 11B, the sensor 1110 may be disposed, for example, in a shoe at a position close to a body measurement location (e.g., sole of the foot or ankle), to obtain data. In this case, the obtained data may be transmitted to the mobile device 1100 through the communication interface, and the processor in the mobile device 1100 may estimate body temperature and output an estimation result to the display as illustrated herein.

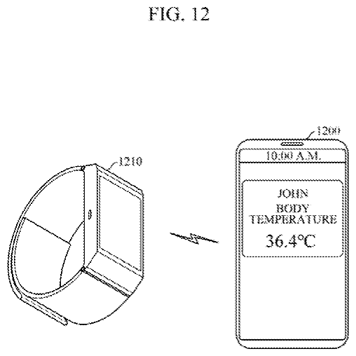

Referring to FIG. 12, the electronic device may be implemented as a combination of a wristwatch-type wearable device and a mobile device such as a smartphone. For example, a memory, a communication interface, and a processor for estimating body temperature may be mounted in a main body of a mobile device 1200. Upon receiving a request for measuring body temperature, the processor of the mobile device 1200 may control the communication interface to communicate with a communication module mounted in a main body of the wearable device 1210, to obtain data through the communication interface. Further, upon receiving data, such as the first temperature, the second temperature, the third temperature, and/or the pulse wave signal, etc., from the wearable device, the processor may estimate body temperature and output an estimation result and information related to body temperature on the display of the mobile device 100 through an output interface as illustrated herein.

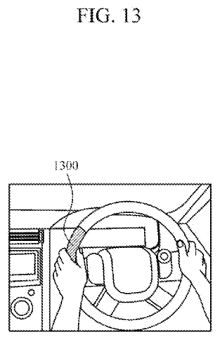

Referring to FIG. 13, an electronic device 1300 may be implemented in a steering wheel of a vehicle.

For example, the electronic device 1300 may be implemented in the vehicle steering wheel that comes into contact with a surface of the palm of a driver's hand, and may estimate the driver's body temperature. In particular, the electronic device 1300 may provide a user with a body temperature estimation result as sound by using an in-vehicle electronic device, or may transmit the estimation result through a communication module, provided in the electronic device 1300, to an external device, e.g., a mobile device, a tablet PC, other medical device, and the like. In addition, the electronic device 1300 may transmit the body temperature estimation result to the in-vehicle electronic device, such that the in-vehicle electronic device may adjust temperature of the vehicle based on the estimation result.

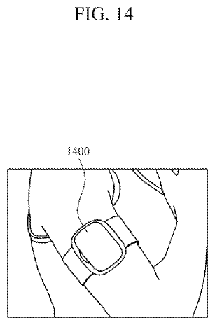

Referring to FIG. 14, an electronic device 1400 may be implemented as a patch-type device.

For example, the electronic device 1400 may be fixed to a body measurement location (e.g., upper arm) by a strap, to measure a user's body temperature. In particular, the electronic device 1400 may provide the user with an estimated body temperature as sound or through a display, or may transmit the estimated body temperature to an external device, e.g., a mobile device, a tablet PC, other medical device, etc., through a communication module provided in the electronic device 1400.

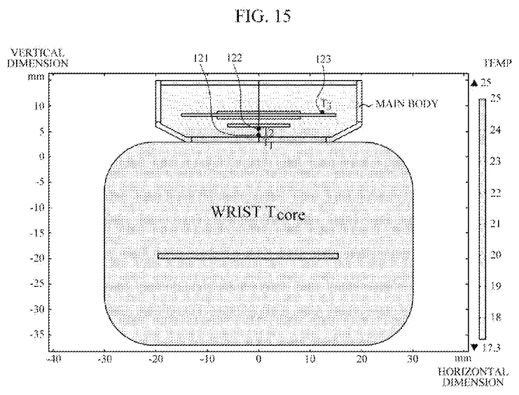

FIG. 15 illustrates a simulation result of estimating an air temperature according to an example embodiment of the present disclosure. Since a body temperature is estimated based on an estimated air temperature and an estimated core body temperature according to embodiments of the disclosure, the more accurate the estimated air temperature is, the more accurate the estimation of the body temperature is.

As shown in FIG. 15, a main body of an electronic device includes an upper surface and a lower surface. When the lower surface is in contact with a user's wrist, the body heat of the user may be conveyed to the lower surface of the main body, and then may transfer from the lower surface to the upper surface of the main body.

Referring to FIG. 15, the main body may include a first temperature sensor 121 and a second temperature sensor 122 that are arranged in a vertical direction (e.g., a thickness direction) of the main body. Also, the main body may include a third temperature sensor 123 which is located farther from the lower surface of the main body (e.g., a contact surface between the main body and the user's wrist) than the first temperature sensor 121 and the second temperature sensor 123. The first temperature sensor 121 may be disposed at a vertical distance (i.e., a thickness direction) of 5 mm or less from the contact surface, and the third temperature sensor 123 may be disposed at a vertical distance of 10 mm or less from the contact surface of the main body 110. In addition, a vertical distance between the first temperature sensor 121 and the second temperature sensor 122 may be 10 mm or less, and a distance between the third temperature sensor 123 and the second temperature sensor 122 may be 10 mm to 50 mm. A first thermally conductive material 210 may be provided between the first temperature sensor 121 and the second thermally conductive material 220, a second thermally conductive material 220 may be provided between the second temperature sensor 122 and the third temperature sensor 123, and a third thermally conductive material 230 may be provided between the third temperature sensor 123 and an upper surface 200 of the main body 110. Each or at least one of the first thermally conductive material 210, the second thermally conductive material 220, and the third thermally conductive material 230 may be insulators having a minimum thickness of 0.4 mm or (i.e., 0.4 mm or greater, and preferably from 0.4 mm to 1.3 mm, and may be materials (e.g., polyurethane foam or air) having a thermal conductivity of 0.1 W/mK or low. Each of the first, second, and third temperatures sensors 121-123 may be set to have a minimum height of 0.3 mm (i.e., 0.3 mm or greater, and preferably from 0.3 mm to 0.5 mm).

When the first temperature sensor 121, the second temperature sensor 122, and the third temperature sensor 123 are arranged as described above, the electronic device may be capable of estimating an air temperature at a high accuracy as shown below:

TABLE 2

| Temperature | Temperature | Actual Air | Estimated Air |
|---|---|---|---|
| Sensor T3 | Sensor T2 | Temperature | Temperature |
| 20.96° C. | 22.62° C. | 15° C. | 14.96° C. |
| 24.77° C. | 26.10° C. | 20° C. | 19.98° C. |
| 29.18° C. | 30.34° C. | 25° C. | 25.00° C. |
| 32.39° C. | 33.05° C. | 30° C. | 30.00° C. |

As shown above, the air temperatures that are estimated according to embodiments of the present disclosure are quite accurate since the differences between the estimated air temperatures and the actual air temperatures are 0.96° C., 0.02° C., 0.00° C., and 0.00° C.

While not restricted thereto, an example embodiment can be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an example embodiment may be written as a computer program transmitted over a computer-readable transmission medium, such as a carrier wave, and received and implemented in general-use or special-purpose digital computers that execute the programs. Moreover, it is understood that in example embodiments, one or more units of the above-described apparatuses and devices can include circuitry, a processor, a microprocessor, etc., and may execute a computer program stored in a computer-readable medium.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An electronic device comprising:
a first temperature sensor configured to measure a first temperature of a surface at a body measurement location of a user;
a second temperature sensor spaced apart from the first temperature sensor and configured to measure a second temperature inside a main body of the electronic device;
a third temperature sensor disposed further away from the first temperature than the second temperature sensor and configured to measure a third temperature inside the main body; and
a processor configured to:
estimate a core temperature at the body measurement location based on the first temperature, the second temperature, and the third temperature;
estimate an ambient temperature outside the main body based on the second temperature and the third temperature; and
estimate a body temperature of the user based on the core temperature at the body measurement location and the ambient temperature outside the main body.

2. The electronic device of claim 1, wherein the processor is further configured to:
obtain a heat loss from a body reference location to the body measurement location, and estimate the body temperature by correcting the core temperature at the body measurement location based on the heat loss.

3. The electronic device of claim 2, wherein the processor is further configured to obtain the heat loss based on a difference between the first temperature and the ambient temperature outside the main body.

4. The electronic device of claim 1, further comprising a pulse wave sensor comprising a light source and a detector, and configured to measure a pulse wave signal of the user,
wherein the processor is further configured to:
estimate a first heat flux based on a difference between the first temperature and the second temperature;
estimate a skin blood flow volume based on the pulse wave signal; and
estimate the core temperature at the body measurement location based on the first heat flux, the first temperature, and the skin blood flow volume.

5. The electronic device of claim 4, the processor is further configured to estimate the core temperature at the body measurement location by combining a ratio between the estimated first heat flux and the skin blood flow volume with the first temperature.

6. The electronic device of claim 1, wherein the processor is further configured to:
estimate a second heat flux based on a difference between the second temperature and the third temperature; and
estimate the ambient temperature outside the main body by combining the second heat flux with the third temperature.

7. The electronic device of claim 6, wherein the processor is further configured to correct the second heat flux based on a resistance value of a thermally conductive material disposed between the second temperature sensor and the third temperature sensor, a resistance value of a thermally conductive material disposed between the third temperature sensor and the surface of the main body, and a resistance value of the surface of the main body.

8. The electronic device of claim 1, wherein at least one of the first temperature sensor, the second temperature sensor, and the third temperature sensor is a thermistor.

9. The electronic device of claim 1, wherein the first temperature sensor is disposed at a vertical distance of 5 mm or less from a contact surface between the main body and the user.

10. The electronic device of claim 1, wherein the third temperature sensor is disposed at a vertical distance of 10 mm or less below the surface of the main body.

11. The electronic device of claim 1, wherein a distance between the first temperature sensor and the second temperature sensor is 10 mm or less, and a distance between the third temperature sensor and the second temperature sensor is 50 mm or less.

12. The electronic device of claim 1, further comprising a display configured to output at least one of the first temperature, the second temperature, the third temperature, the core temperature at the body measurement location, and the ambient temperature outside the main body.

13. A method of estimating body temperature using an electronic device, the method comprising:
by using a first temperature sensor, measuring a first temperature of a surface at a body measurement location;
by using a second temperature sensor spaced apart from the first temperature sensor, measuring a second temperature inside a main body of the electronic device;
by using a third temperature sensor disposed further away from the first temperature than the second temperature sensor, measuring a third temperature inside the main body;
estimating a core temperature at the body measurement location based on the first temperature and the second temperature;
estimating an ambient temperature outside the main body based on the second temperature and the third temperature; and
estimating a body temperature of a user based on the core temperature at the body measurement location and the ambient temperature outside the main body.

14. The method of claim 13, wherein the estimating of the body temperature of the user comprises:
obtaining a heat loss from a body reference location to the body measurement location; and
estimating the body temperature by correcting the core temperature at the body measurement location based on the heat loss.

15. The method of claim 14, wherein the estimating of the body temperature of the user comprises:
obtaining the heat loss based on a difference between the first temperature and the ambient temperature outside the main body.

16. The method of claim 13, wherein the estimating of the core temperature at the body measurement location comprises:
estimating a first heat flux based on the first temperature and the second temperature; and
estimating the core temperature at the body measurement location based on the first heat flux, the first temperature, and a skin blood flow volume measured by a pulse wave sensor.

17. The method of claim 13, wherein the estimating of the ambient temperature outside the main body comprises:
estimating a second heat flux based on a difference between the second temperature and the third temperature; and
estimating the ambient temperature outside the main body by combining the second heat flux with the third temperature.

18. The method of claim 17, wherein the estimating of the ambient temperature outside the main body comprises:
correcting the second heat flux based on a resistance value of a thermally conductive material disposed between the second temperature sensor and the third temperature sensor, a resistance value of a thermally conductive material disposed between the third temperature sensor and the surface of the main body, and a resistance value of the surface of the main body.

19. A wearable device comprising:
a main body; and
a strap connected to the main body,
wherein the main body comprises:
a first temperature sensor configured to measure a skin temperature of a user, a second temperature sensor configured to measure a first internal temperature of the main body, a third temperature sensor configured to a second internal temperature of the main body, wherein the second temperature sensor is disposed between the first temperature sensor and the third temperature sensor in a thickness direction of the main body; and
a processor configured to estimate a core temperature at a body measurement location based on the skin temperature and the first internal temperature, estimate an ambient temperature outside the main body based on the first internal temperature and the second internal temperature of the main body, and estimate a body temperature of the user based on the core temperature at the body measurement location and the ambient temperature outside the main body.

20. The wearable device of claim 19, further comprising:
a display;
a sensor circuit board to which the first temperature sensor and a PPG sensor are connected; and
a main circuit board to which the processor is connected and disposed between the sensor circuit board and the display,
wherein the second temperature sensor is disposed between the sensor circuit board and the main circuit board, and the third temperature sensor is disposed between the main circuit board and the display.
